(19) United States
(12) Patent Application Publication
(10) Pub. No.: US 2013/0303464 A1
(43) Pub. Date: Nov. 14, 2013

(54) STABLE READY-TO-USE CETRORELIX INJECTION

(71) Applicants: Pankaj Patel, Ahmedabad (IN); Bhavesh Patel, Ahmedabad (IN); Ashish Sehgal, Ahmedabad (IN); Jayanta Kumar Mandal, Ahmedabad (IN)

(72) Inventors: Pankaj Patel, Ahmedabad (IN); Bhavesh Patel, Ahmedabad (IN); Ashish Sehgal, Ahmedabad (IN); Jayanta Kumar Mandal, Ahmedabad (IN)

(73) Assignee: Astron Research Limited, Gujarat (IN)

(21) Appl. No.: 13/991,325

(22) PCT Filed: Dec. 5, 2011
(86) PCT No.: PCT/IN11/00825

§ 371 Date: Jul. 19, 2013

(30) Foreign Application Priority Data
Dec. 6, 2010 (IN) 3306/MUM/2010

(51) Int. Cl. A61K 38/08

(52) U.S. Cl. CPC A61K 38/08
USPC 514/21.6

(57) ABSTRACT

The present invention discloses a stable ready-to-use aqueous pharmaceutical preparation containing Cetrorelix or its pharmaceutically acceptable salt, wherein the preparation does not contain any surfactant. Further, the present invention discloses process for the preparation of said stable ready-to-use aqueous pharmaceutical preparation.

### FIELD OF THE INVENTION

The present invention relates to a stable aqueous pharmaceutical preparation containing Cetrorelix or its pharmaceutically acceptable salt in the form of ready-to-use solutions avoiding reconstitution step prior to use and process for preparing such preparations.

### BACKGROUND-ART

Chemically, Cetrorelix is gonadotropin releasing hormone antagonist (GnRH antagonist) acetyl-D-3-(2′-naphtyl)-alanine-D-4-chlorophenylalanine-D-3-(3′-pyridyl)-alanine-L-serine-L-tyrosine-D-citruline-L-leucine-L-arginine-L-proline-D-alanine-amide (C₇₀H₉₂ClN₁₇O₁₄) having the following formula:

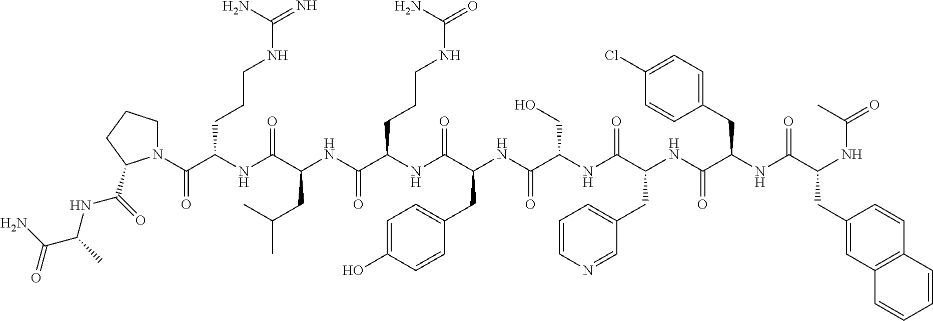 [SMILES: CC(=O)N[C@H](Cc1ccc2ccccc2c1)C(=O)N[C@H](Cc1ccc(Cl)cc1)C(=O)N[C@H](Cc1cccnc1)C(=O)N[C@@H](CO)C(=O)N[C@@H](Cc1ccc(O)cc1)C(=O)N[C@H](CCCNC(N)=O)C(=O)N[C@@H](CC(C)C)C(=O)N[C@@H](CCCNC(=N)N)C(=O)N1CCC[C@H]1C(=O)N[C@H](C)C(N)=O]

Cetrorelix is a decapeptide with a terminal acid amide group. Cetrorelix was earlier disclosed in U.S. Pat. No. 4,800,191 patent. Cetrorelix is currently been marketed as a Cetrorelix acetate formulated as a lyophilized formulation (Cetrotide®) by Merck Serono and Company for the inhibition of premature LH surges in women undergoing controlled ovarian stimulation. Cetrotide® is currently available in two presentations, i.e. as a lyophilisate of 3 mg of Cetrorelix with either 1 ml or 3 ml of water for reconstitution in a prefilled syringe.

Cetrorelix is used to treat hormone-sensitive cancers of the prostate and breast (in pre-/perimenopausal women) and some benign gynaecological disorders. The drug works by blocking the action of GnRH upon the pituitary, thus rapidly suppressing the production and action of LH and FSH.

Formulation aspect of developing a stable aqueous solution of Cetrorelix acetate is hindered by the known fact that oligopeptides particularly having a terminal acid amide group are prone to gel formation.

Patent CA2115943 discloses the development proceedings of the marketed Cetrorelix injection. The patent discloses that aqueous solutions of the decapeptide can not be autoclaved because these aqueous solutions of the decapeptide are unstable. Further, with conventional sterilization at prescribed conditions the decapeptide tends to decompose; hence to obtain an injectable composition it was necessary to develop a lyophilisate composition.

CA2115943 also discloses that bulking agent is necessary in the lyophilisate composition of Cetrorelix to obtain a stable cake. Particularly useful bulking agent used in the lyophilisate composition is mannitol.

CA2115943 also discloses that oligopeptides tend to form gels. During sterile filtration of the bulk solution, formation of gels in the sterile filter would increase the viscosity of the solution and hence hinder the filtration step. In order to overcome the problems associated with sterile filtration, it was found that acidification with acetic acid showed promising results. Hence, for the preparation of sterile Cetrorelix lyophilisate, Cetrorelix was dissolved in 30% v/v of acetic acid, the obtained solution was further diluted with water, a bulking agent was dissolved and the obtained solution was sterilized by filtration. The obtained sterilized solution was filled into suitable container and lyophilized.

It was observed that the bulk solution would have a pH of 2.47 and osmolality of about 675 mosmol/kg. This bulk solution cannot be administered as a ready-to-use injection solution as such because of the following limitations:
hypertonic nature of the solution which may cause local site eg., irritation, edema, swellings, redness etc.
quantity of acetic acid is above safety level for subcutaneous/parenteral route of administration.

U.S. Pat. No. 7,718,599 claims a pharmaceutical composition for parenteral administration, which contains Cetrorelix acetate in a concentration of 2.5 mg/ml and comprises a pharmaceutically acceptable acid selected from a group of gluconic acid, glucaric acid or galactouronic acid and is capable of imparting a pH in between 2.5 to 4.5 to the composition which helps in suppressing aggregation of Cetrorelix acetate.

U.S. Pat. No. 7,214,662 focuses on an aqueous solution of 500 mg of LHRH antagonist (Cetrorelix), wherein the composition comprises of an acid selected from a group of carboxylic acid, gluconic acid, hydrocarboxylic acid and gluconic acid deltalactone in combination with a surfactant Tween 80; which improves the solubility of the LHRH antagonist. Further, the patent also mentions that the use said acid and surfactant reduces the tendency of LHRH substances to aggregate.

Marketed Cetrorelix acetate injection (Cetrotide®) is available in lyophilized form which when reconstituted with water for injection provides a clear colorless solution.

Following are the disadvantages which may be associated with lyophilized dosage forms:
high manufacturing cost and complexity of equipments
needs an additional step of reconstitution prior to administration
improper reconstitution may sometimes results in failure to provide a clear solution.

Considering the issues disclosed in the back-ground art related with the formulation aspects of Cetrorelix product, a need exists for providing a stable Cetrorelix aqueous solution as a pharmaceutical preparation which can be administered parenterally as a ready-to-use option to a person in need thereof, wherein the pharmaceutical preparation can be conveniently prepared and sterilized by filtration, without the hassles of reconstitution.

With the efforts of the inventors of the current invention, a stable aqueous ready-to-use Cetrorelix solution for parenteral administration has been developed, which would overcome the formulation aspect issues as disclosed in the back-ground art.

### OBJECTS OF THE INVENTION

The main object of the invention is to provide a stable ready-to-use aqueous pharmaceutical preparation containing Cetrorelix or its pharmaceutically acceptable salt for parenteral administration, wherein the said preparation does not comprise of a surfactant.

Another object of the present invention is to provide a stable ready-to-use aqueous pharmaceutical preparation containing Cetrorelix or its pharmaceutically acceptable salt for parenteral administration, wherein the said preparation does not comprise of a surfactant and wherein the concentration of Cetrorelix is 0.25 mg/ml or more and have a pH in between 2.5 to 5.

Still another object of this invention is to provides a process for manufacturing a stable ready-to-use aqueous pharmaceutical preparation of Cetrorelix or its pharmaceutically acceptable salt for parenteral administration, wherein the said preparation does not comprise of a surfactant and wherein the concentration of Cetrorelix is 0.25 mg/ml or more and have a pH in between 2.5 to 5.

### SUMMARY OF THE INVENTION

It has been found out surprisingly by the inventors of the present invention that a stable ready-to-use aqueous pharmaceutical preparation containing Cetrorelix or its pharmaceutically acceptable salt for parenteral administration, can be prepared by using low amounts of glacial acetic acid with Cetrorelix acetate, tonicity adjusting agent and optionally other pharmaceutically acceptable excipients in water.

### DETAILED DESCRIPTION

Present invention relates to a stable ready-to-use aqueous pharmaceutical preparation of Cetrorelix or its pharmaceutically acceptable salt for parenteral administration, wherein the said preparation does not comprise of a surfactant and wherein the concentration of Cetrorelix is 0.25 mg/ml or more and has a pH in between 2.5 to 5.

Pharmaceutical preparation according to the present invention is administered parenterally, wherein the preferred mode of parenteral administration is subcutaneous administration. Other modes of parenteral administration of the pharmaceutical preparation may include intravenous administration, intramuscular administration, etc.

According to the present invention, use of low amount of glacial acetic acid in the pharmaceutical preparation corresponds to around 0.1% w/v to 0.5% w/v of the said preparation.

Use of the said low amounts of glacial acetic acid in the said pharmaceutical preparation would help in providing following advantages to the preparation:
desirable isotonicity,
absence of formation of aggregates of Cetrorelix acetate,
filter sterilization,
pH in between 2.5 to 5,
acceptable level/amounts of acetic acid as per as requirement for parenteral preparations.

Further, according to the present invention, the stable ready-to-use aqueous pharmaceutical preparation of Cetrorelix or its pharmaceutically acceptable salt for parenteral administration does not comprise of any surfactant.

According to the present invention, the active used in the said pharmaceutical preparation comprises of Cetrorelix or its pharmaceutically acceptable salt in the form of acetate. The said pharmaceutical preparation comprises of Cetrorelix or its pharmaceutically acceptable salt in proportion of 0.025% w/v or more. In the said pharmaceutical preparation, the concentration of Cetrorelix or its pharmaceutically acceptable salt is in an amount of 0.25 mg/ml or more; preferably in a range of 0.25 to 0.75 mg/ml, more preferably in a range of 0.25 to 0.5 mg/ml. Further, variation in an amount of the active in the said pharmaceutical preparation is possible which would be obvious to a person skilled in the art.

Further, according to the present invention, the pH of the said pharmaceutical preparation is in the range of 2.5 to 5; preferably in the range of 2.8 to 3.5.

According to the present invention, the pharmaceutical preparation comprise of tonicity adjusting agents. Tonicity adjusting agents decrease the hemolysis of blood cells and reduce pain and irritation at the injection site. Tonicity adjusting agents that can be included in the said pharmaceutical preparation comprise of mannitol, lactose, dextrose, or the likes thereof. Preferred tonicity adjusting agent for the said pharmaceutical preparation is mannitol.

According to the present invention, the amount of tonicity adjusting agent used in the said preparation is adjusted to obtain osmolality of the said preparation in the range of 290 to 330 mOsm/kg. An osmometer can be used to check and adjust the amount of tonicity adjusting agent to be added to obtain the desired osmolality.

Further, according to the present invention, other optional pharmaceutical excipients which can be used in the said pharmaceutical preparation are chelating agents, buffers and pH adjusters, antioxidants and reducing agents, antimicrobial preservatives, etc.

The present invention also provides a process for the manufacture of a stable ready-to-use aqueous pharmaceutical preparation of Cetrorelix or its pharmaceutically acceptable salt for parenteral administration. A generalized process for the manufacture of the said pharmaceutical preparation of Cetrorelix or its pharmaceutically acceptable salt according to the present invention comprises of:
dissolving glacial acetic acid, Cetrorelix or its pharmaceutically acceptable salt and tonicity adjusting agent in water to obtain a solution,
if required, make up the volume with water to obtain clear, isotonic, iso-osmolar aqueous pharmaceutical preparation,
filter sterilize the pharmaceutical preparation and fill in suitable container/closure system.

Optional inert gas sparging (nitrogen gas) can be carried out during any of the steps of the process. Modifications in the generalized process can be made as known to the person skilled in the art.

Pharmaceutical preparation prepared according to the process disclosed in the present invention can be conventionally sterilized using filter sterilization, e.g. 0.2 micron filter, to render the solution sterile. This sterile pharmaceutical preparation is filled in suitable container/closure system, e.g., ampoules, vials, prefilled syringe system, etc.

According to the present invention, the said pharmaceutical preparation can be directly filled preferably in a prefilled syringe system. The advantages associated with the prefilled syringe system are:
preferred by physicians/health-care professionals due to ease of handling,
elimination of dosing error.

According to the present invention, the said pharmaceutical preparation is stable; wherein “stable pharmaceutical preparation” is defined as no aggregation observed when the said pharmaceutical preparation is kept for stability studies carried out at 2° C. to 8° C. (Real time study) and 25° C./60% relative humidity (Accelerated study) for at least 6 months and wherein the assay of Cetrorelix would not be less than 90%.

The assay of Cetrorelix in the said pharmaceutical preparation can be carried out by any of the methods known to the person skilled in the art, e.g. High performance liquid chromatography (HPLC method), Spectrophotometry (UV spectrophotometry), etc. According to the present invention, HPLC was used for performing the assay studies.

### EXAMPLE

The present invention has been described by way of example only, and it is to be recognized that modifications thereto falling within the scope and spirit of the appended claims, and which would be obvious to a person skilled in the art based upon the disclosure herein, are also considered to be included within the scope of this invention.

### Example 1

### Composition

### Each ml Contains

Cetrorelix acetate eq. to Cetrorelix . . . 0.25 mg

### Mannitol . . . 45.54 mg

Glacial acetic acid . . . 3.0 mg
Water for injection . . . q.s. 1 ml

### Manufacturing Process:

a) Transfer approximately 85% of water for injection (20-25° C.) into Stainless Steel (S.S) manufacturing vessel. Sparge nitrogen gas into the water for injection for 15 minutes (Solution A).
b) Separately prepared approximately 25% w/v of glacial acetic acid solution in water for injection. Add Cetrorelix acetate and stir till it dissolves completely (Solution B).
c) Solution B is added into Solution A and stirred for 10 minutes.
d) Add mannitol and stir to dissolve it completely.
e) Make up the volume with water for injection.
f) Filter the solution through sterile 0.2 micron filter to render the solution sterile.
g) Fill the sterile bulk solution into suitable container/closure system.

### Observation:

The bulk solution is clear, colorless with no filterability issue observed because of addition of glacial acetic acid which prevents gel formation completely.

No aggregation was observed on stability at 2-8° C. and 25° C./60% relative humidity for at least 6 months.

### Stability Data:

| | | | 1 M | 2 M | 3 M | 6 M | |
|---|---|---|---|---|---|---|---|
| | Initial | | 25° C./ | 25° C./ | 25° C./ | 25° C./ | |
| Parameters | Unfiltered | Filtered | 60% RH | 60% RH | 60% RH | 60% RH | 2-8° C. |
| Description | CCS | CCS | CCS | CCS | CCS | CCS | CCS |
| pH | 3.09 | 3.05 | 3.01 | 3.03 | 3.05 | 3.06 | 3.07 |
| Assay of Cetrorelix | 98.9 | 98.7 | 98.0 | 98.0 | 97.2 | 95.7 | 96.6 |

Related substances

| | | | | | | | |
|---|---|---|---|---|---|---|---|
| Single max. impurity | 0.235 | 0.23 | 0.24 | 0.25 | 0.52 | 1.00 | 0.16 |
| Total impurity | 0.286 | 0.284 | 0.50 | 0.55 | 0.93 | 1.59 | 0.27 |

| | |
|---|---|
| Osmolality | 317 mOsm/kg |

CCS—Clear colorless solution
NA—Not Analyzed

### Example 2

### Composition

### Each ml Contains

Cetrorelix acetate eq. to Cetrorelix . . . 0.5 mg

### Mannitol . . . 40.91 mg

Glacial acetic acid . . . 4.5 mg
Water for injection . . . q.s. 1 ml

### Manufacturing Process:

a) Transfer approximately 85% of water for injection (20-25° C.) into Stainless Steel (S.S) manufacturing vessel. Sparge nitrogen gas into the water for injection for 15 minutes (Solution A).
b) Separately prepared approximately 33% w/v of glacial acetic acid solution in water for injection. Add Cetrorelix acetate and stir till it dissolves completely (Solution B).
c) Solution B is added into Solution A and stirred for 10 minutes.
d) Add mannitol and stir to dissolve it completely.
e) Make up the volume with water for injection.
f) Filter the solution through sterile 0.2 micron filter to render the solution sterile.
g) Fill the sterile bulk solution into suitable container/closure system.

### Observation:

The bulk solution is clear, colorless with no filterability issue observed because of addition of glacial acetic acid which prevents gel formation completely.

No aggregation was observed on stability at 2-8° C. and 25° C./60% relative humidity for at least 6 months.

### Stability Data:

| | | | 1 M | 2 M | 3 M | 6 M | |
|---|---|---|---|---|---|---|---|
| | Initial | | 25° C./ | 25° C./ | 25° C./ | 25° C./ | |
| Parameters | Unfiltered | Filtered | 60% RH | 60% RH | 60% RH | 60% RH | 2-8° C. |
| Description | CCS | CCS | CCS | CCS | CCS | CCS | CCS |
| pH | 3.25 | 3.23 | 3.27 | 3.27 | 3.22 | 3.06 | 3.07 |
| Assay of Cetrorelix | 107.0 | 106.8 | 106.0 | 104.7 | 1.4.0 | 101.2 | 104.0 |

Related substances

| | | | | | | | |
|---|---|---|---|---|---|---|---|
| Single max. impurity | 0.37 | 0.17 | 0.22 | 0.43 | 0.59 | 0.99 | 0.2 |
| Total impurity | 1.26 | 0.58 | 1.00 | 1.35 | 1.54 | 2.14 | 0.82 |

| | |
|---|---|
| Osmolality | 318 mOsm/kg |

CCS—Clear colorless solution
NA—Not Analyzed

### CONCLUSION

The results of the studies performed for Example 1 and 2 according to the present invention, disclose that the ready-to-use aqueous pharmaceutical preparations of Cetrorelix are stable and can be administered to a person in need thereof.

1. A stable ready-to-use aqueous pharmaceutical preparation of Cetrorelix for parenteral administration, wherein the preparation comprises Cetrorelix or its pharmaceutically acceptable salt in an amount of 0.025% w/v or more, low amounts of glacial acetic acid, a tonicity adjusting agent, and optionally other pharmaceutically acceptable excipients, dissolved in water; wherein the preparation does not comprise a surfactant.

2. The pharmaceutical preparation as claimed in claim 1, wherein the pharmaceutically acceptable salt of Cetrorelix is Cetrorelix acetate.

3. The pharmaceutical preparation as claimed in claim 1, wherein the concentration of Cetrorelix or its pharmaceutically acceptable salt is 0.025 to 0.075% w/v of the preparation.

4. The pharmaceutical preparation as claimed in claim 1, wherein low amounts of glacial acetic acid corresponds to about 0.1 to 0.5% w/v of the preparation.

5. The pharmaceutical preparation as claimed in claim 1, wherein the pH of the preparation is in between 2.5 to 5.

6. The pharmaceutical preparation as claimed in claim 1, wherein tonicity adjusting agent is selected from the group consisting of mannitol, lactose, and dextrose, and the osmolality of the preparation is in between 290 to 330 mOsm/kg.

7. A process for the manufacture of a stable ready-to-use aqueous pharmaceutical preparation of Cetrorelix for parenteral administration, wherein the steps comprise of:
a) dissolving glacial acetic acid, Cetrorelix or its pharmaceutically acceptable salt, and a tonicity adjusting agent in water to obtain a solution,
b) filtering the obtained solution through sterile filter, and
c) filling the sterile Cetrorelix preparation in a suitable container/closure system.

8. The process of claim 7, wherein the pH of the preparation is in the range in between 2.5 to 5; and the osmolality of the preparation is in between 290 to 330 mOsm/kg.

9. A stable ready-to-use aqueous pharmaceutical preparation of Cetrorelix for parenteral administration, comprising:
Cetrorelix acetate . . . 0.25 mg;
Mannitol . . . q.s. to 290 to 330 mOsm/kg;
Glacial acetic acid . . . 3.0 mg; and
Water for injection . . . q.s. 1 ml.

10. A stable ready-to-use aqueous pharmaceutical preparation of Cetrorelix for parenteral administration, comprising:
Cetrorelix acetate . . . 0.5 mg;
Mannitol . . . q.s. to 290 to 330 mOsm/kg;
Glacial acetic acid . . . 4.5 mg; and
Water for injection . . . q.s. 1 ml.

11. The pharmaceutical preparation of claim 2, wherein the concentration of Cetrorelix or its pharmaceutically acceptable salt is 0.025 to 0.075% w/v of the preparation.

12. The pharmaceutical preparation of claim 11, wherein the concentration of Cetrorelix or its pharmaceutically acceptable salt is 0.025 to 0.05% w/v of the preparation.

13. The pharmaceutical preparation of claim 3, wherein the concentration of Cetrorelix or is pharmaceutically acceptable salt is 0.025 to 0.05% w/v of the preparation.

14. The pharmaceutical preparation as claimed in claim 5, wherein the pH of the preparation is between 2.8 to 3.5.

15. The process of claim 8, wherein the pH of the preparation is between 2.8 to 3.5.
